(19) United States
(12) Patent Application Publication
(10) Pub. No.: US 2025/0087356 A1
(43) Pub. Date: Mar. 13, 2025

(54) Computer Implemented Method for Classification of a Medical Relevance of a Deviation Between Cardiac Current Curves, Training Method and System

(71) Applicant: BIOTRONIK SE & Co. KG, Berlin (DE)

(72) Inventors: Bjoern Henrik DIEM, Berlin (DE); Antje LINNEMANN, Berlin (DE); Anastasia REICH, Berlin (DE)

(73) Assignee: BIOTRONIK SE & Co. KG, Berlin (DE)

(21) Appl. No.: 18/727,252

(22) PCT Filed: Dec. 15, 2022
(86) PCT No.: PCT/EP2022/086036

§ 371 Date: Jul. 8, 2024

(30) Foreign Application Priority Data
Jan. 14, 2022 (EP) 22151558.8

(51) Int. Cl. G16H 50/20; A61B 5/349; G16H 10/60

(52) U.S. Cl. CPC G16H 50/20; A61B 5/349; G16H 10/60

(57) ABSTRACT

A computer-implemented method for classification of a medical relevance of a deviation between cardiac current curves, comprising applying a machine learning algorithm to the pre-acquired first cardiac current curve data and the pre-acquired at least second cardiac current curve data for classification of the medical relevance of the deviation between the pre-acquired first cardiac current curve data and the pre-acquired at least second cardiac current curve data. Furthermore, a corresponding system and a method for providing a trained machine learning algorithm are provided.

### CROSS-REFERENCE TO RELATED APPLICATIONS

This application is the United States National Phase under 35 U.S.C. § 371 of PCT International Patent Application No. PCT/EP2022/086036, filed on Dec. 15, 2022, which claims the benefit of European Patent Application No. 22151558.8, filed on Jan. 14, 2022, the disclosures of which are hereby incorporated by reference herein in their entireties.

### TECHNICAL FIELD

The present invention relates to a computer-implemented method for classification of a medical relevance of a deviation between cardiac current curves.

### BACKGROUND

Furthermore, the present invention relates to a computer implemented method for providing a trained machine learning algorithm configured to classify a medical relevance of a deviation between cardiac current curves.

In addition, the present invention relates to a system for classification of a medical relevance of a deviation between cardiac current curves.

Many diseases of the heart are accompanied by changes in the ECG. These could be detected at an early stage by close-meshed ECG checks. However, this is logistically not feasible in everyday life.

Conventionally, said ECG is recorded at after care visits of the patient having an implantable medical device at a health provider, such after care visits typically being scheduled every 1 to 12 months. To this end, a twelve-channel ECG is recorded at the health provider's site. The recording of a conventional twelve-channel ECG is however associated with a relevant expenditure of time and personnel. Alternatively, remote transmission of a twelve-channel ECG requires the active cooperation and compliance of the patient, who may be overtaxed.

The present disclosure is directed toward overcoming one or more of the above-mentioned problems, though not necessarily limited to embodiments that do.

### SUMMARY

It is therefore an object of the present invention to provide an improved method for automated remote monitoring of cardiac current curves for new onset or progression of heart disease with higher frequency than possible by outpatient follow-up with alerting of the attending physician in case of change.

At least the above object is solved by a computer implemented method for classification of a medical relevance of a deviation between cardiac current curves having the features of claim 1.

Furthermore, at least the above object is solved by a computer implemented method for providing a trained machine learning algorithm configured to classify a medical relevance of a deviation between cardiac current curves having the features of claim 13.

In addition, at least the above object is solved by a system for classification of a medical relevance of a deviation between cardiac current curves having the features of claim 14.

Further developments and advantageous embodiments are defined in the dependent claims.

The present invention provides a computer implemented method for classification of a medical relevance of a deviation between cardiac current curves. The method comprises providing a first data set comprising first cardiac current curve data of a patient acquired during a first time interval and at least second cardiac current curve data of the patient acquired during a second time interval by an implantable medical device, said first time interval and said second time interval differing from each other.

Furthermore, the method comprises applying a machine learning algorithm to the pre-acquired first cardiac current curve data and the pre-acquired at least second cardiac current curve data for classification of the medical relevance of the deviation between the pre-acquired first cardiac current curve data and the pre-acquired at least second cardiac current curve data.

Moreover, the method comprises outputting a second data set comprising at least a first class representing a medically relevant deviation between the first cardiac current curve data and the second cardiac current curve data and/or a second class representing a medically not relevant deviation or no deviation between the first cardiac current curve data and the second cardiac current curve data.

Furthermore, the present invention provides a computer implemented method for providing a trained machine learning algorithm configured to classify a medical relevance of a deviation between cardiac current curves.

The method comprises receiving a first training data set comprising first cardiac current curve data of a patient acquired during a first time interval and at least second cardiac current curve data of the patient acquired during a second time interval by an implantable medical device, said first time interval and said second time interval differing from each other.

In addition, the method comprises receiving a second training data set comprising at least a first class representing a medically relevant deviation between the first cardiac current curve data and the second cardiac current curve data and/or a second class representing a medically not relevant deviation or no deviation between the first cardiac current curve data and the second cardiac current curve data.

Furthermore, the method comprises training the machine learning algorithm by an optimization algorithm which calculates an extreme value of a loss function for classification of the first class representing a medically relevant deviation between the first cardiac current curve data and the second cardiac current curve data and/or the second class representing a medically not relevant deviation or no deviation between the first cardiac current curve data and the second cardiac current curve data from the first cardiac current curve data and the second cardiac current curve data.

Moreover, the present invention provides a system for classification of a medical relevance of a deviation between cardiac current curves. The system comprises an implantable medical device for providing a first data set comprising first cardiac current curve data of a patient acquired during a first time interval and at least second cardiac current curve data of the patient acquired during a second time interval, said first time interval and said second time interval differing from each other.

Moreover, the system comprises means for applying a machine learning algorithm to the pre-acquired first cardiac current curve data and the pre-acquired at least second cardiac current curve data for classification of the medical relevance of the deviation between the pre-acquired first cardiac current curve data and the pre-acquired at least second cardiac current curve data.

In addition, the system comprises means for outputting a second data set comprising at least a first class representing a medically relevant deviation between the first cardiac current curve data and the second cardiac current curve data and/or a second class representing a medically not relevant deviation or no deviation between the first cardiac current curve data and the second cardiac current curve data.

An idea of the present invention is to provide automatic remote monitoring of cardiac current curves for new onset or progression of heart disease with higher frequency than possible by outpatient follow-up with alerting of the attending physician in case of change.

Specifically, the present invention is based on the regular recording and transmission of cardiac waveforms to a central server, which then automatically compares the current cardiac waveform with previous cardiac waveforms using a machine learning algorithm, taking into account other implant data and implant settings, and alerts the physician in the event of relevant deviations.

Thus, an improvement of therapy quality through early detection of new onset or worsening of cardiac diseases by means of automatic remote monitoring can be provided.

Machine learning algorithms are based on using statistical techniques to train a data processing system to perform a specific task without being explicitly programmed to do so. The goal of machine learning is to construct algorithms that can learn from data and make predictions. These algorithms create mathematical models that can be used, for example, to classify data or to solve regression type problems.

According to an aspect of the present invention, the first class representing the medically relevant deviation between the first cardiac current curve data and the second cardiac current curve data comprises a plurality of subclasses each representing a medical indication, in particular a cardiac disorder. Thus, advantageously a plurality of different medical conditions can be detected based on the differences of the respective cardiac current curves.

According to a further aspect of the present invention, the second class representing a medically not relevant deviation or no deviation between the first cardiac current curve data and the second cardiac current curve data comprises changes in position, respiration and/or physical exertion. As a result, said medically not relevant deviations can be distinguished from medically relevant deviations.

According to a further aspect of the present invention, the second data set further comprises a third class representing erroneous cardiac current curve data not suitable for application of the machine learning algorithm for classification of the medical relevance of the deviation between cardiac current curves. By classifying erroneous cardiac current curve data in a separate class, false classifications in the first and second classes can be advantageously prevented.

According to a further aspect of the present invention, the third class further represents a case in which there is no deviation between the first cardiac current curve data and the second cardiac current curve data occurs. The machine learning algorithm that advantageously also covers the case of no deviation occurring, which does not fall within the first and second classes.

According to a further aspect of the present invention, if the deviation between the pre-acquired first cardiac current curve data and the pre-acquired at least second cardiac current curve data is classified to be a medically relevant deviation according to the first class, a notification is sent to a communication device of a health care provider. Thus, the healthcare provider is advantageously informed of new onset or progression of heart disease with higher frequency than possible by outpatient follow-up.

According to a further aspect of the present invention, the medically relevant deviation between the pre-acquired first cardiac current curve data and the pre-acquired at least second cardiac current curve data comprises changes in P waves, PQ segment, QRS complex, J point, ST segment, T waves, U waves, TP respectively UP segment and/or a QRS morphology for ischemia, infarction and/or conduction disorders. Theses sections of the cardiac current curve can thus serve as medical parameters usable as input data to the machine learning algorithm.

The P wave is the first wave of a normal cardiac cycle and reflects the depolarization of the right and left atrium of the heart. The PQ segment is the segment between the end of the P wave and the begin of the QRS complex.

The QRS complex is the combination of three of the graphical deflections seen on a typical electrocardiogram. It is usually the central and most visually obvious part of the tracing. It corresponds to the depolarization of the right and left ventricles of the heart followed by the contraction of the large ventricular muscles. In adults, the QRS complex normally lasts 80 ms to 100 ms. The Q, R, and S waves occur in rapid succession, do not all appear in all leads, and reflect a single event and thus are usually considered together. A Q wave is any initial downward deflection. The upward deflections are called R, R′, R″, and the downward deflection after the first R are called S, S′, S″.

The ST segment is the segment between end of the QRS complex (also known as the J point) and the being of the T wave. The T wave represents the repolarization of the ventricles of the heart, and in some cases, an additional U wave follows the T wave. The TP respectively the UP segment closes the cycle.

According to a further aspect of the present invention, the first data set further comprises third cardiac current curve data not originating from the patient from which the first cardiac current curve data and the second cardiac current curve data are collected. Said additional data can advantageously contribute to a more accurate classification of the deviation between the pre-acquired first cardiac current curve data and the pre-acquired at least second cardiac current curve data.

According to a further aspect of the present invention, the first data set further comprises additional medical parameters comprising a patient activity, a thoracic impedance and/or electrode readings (e.g., pacing threshold, sensing amplitude, impedance) of the implantable medical device. Adding said medical parameters advantageously improves a detection accuracy for the first and second class classified by the machine learning algorithm.

According to a further aspect of the present invention, the first cardiac current curve data and the second cardiac current curve data comprise a subcutaneous ECG, in particular a wide-field ECG between electrodes and a housing of the implantable medical device, a pseudo-ECG between a shock coil and the implantable medical device and/or intracardiac current waveforms. It is therefore advantageously not necessary for the patient to perform a multi-channel ECG in a clinical setting.

According to a further aspect of the present invention, the cardiac current curve data is acquired by the implantable medical device at predetermined intervals and/or on request, and wherein the cardiac current curve data is transmitted to a central server via a patient communication device or smartphone. The output data of the algorithm can thus be transmitted to the server for further evaluation according to the predetermined intervals and/or on request thus significantly shortening the time to potentially detect new onset or progression of heart disease with higher frequency than possible by outpatient follow-up with alerting of the attending physician in case of change.

According to a further aspect of the present invention, a beginning of the first time interval differs from a beginning of the second time interval and/or the first time interval ends before a beginning of the second time interval. It can thus be insured that the first cardiac current curve data and the second cardiac current curve data are distinct data sets suitable for determining a deviation between them.

Furthermore, a computer program is disclosed with program code to perform the above defined method(s) when the computer program is executed on a computer. Alternatively or additionally, a computer program is disclosed comprising instructions which, when executed by a processor, cause the processor to perform the steps of the above defined method(s). Accordingly, a computer readable data carrier storing such computer program is described.

The herein described features of the system for classification of a medical relevance of a deviation between cardiac current curves are also disclosed for the computer implemented method for classification of a medical relevance of a deviation between cardiac current curves and vice versa.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

For a more complete understanding of the present invention and advantages thereof, reference is now made to the following description taken in conjunction with the accompanying drawings. The present invention is explained in more detail below using exemplary embodiments, which are specified in the schematic figures of the drawings, in which:

FIG. 1 shows a flowchart of a computer implemented method and system for classification of a medical relevance of a deviation between cardiac current curves according to a preferred embodiment of the present invention; and

FIG. 2 shows a flowchart of a computer implemented method for providing a trained machine learning algorithm configured to classify a medical relevance of a deviation between cardiac current curves according to the preferred embodiment of the present invention.

### DETAILED DESCRIPTION

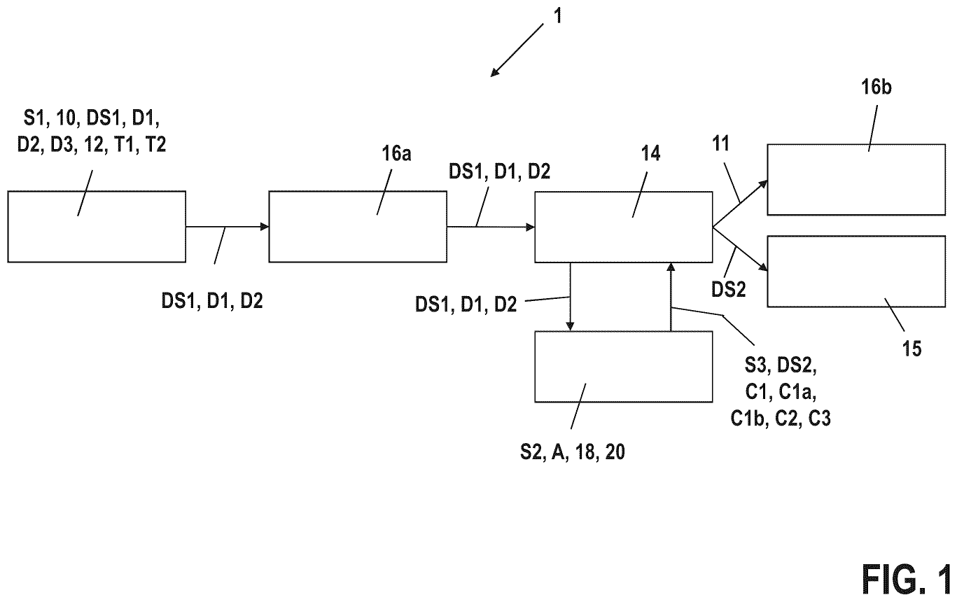

The system 1 shown in FIG. 1 for classification of a medical relevance of a deviation between cardiac current curves comprises an implantable medical device 10 for providing a first data set DS1 comprising first cardiac current curve data D1 of a patient acquired during a first time interval T1 and at least second cardiac current curve data D2 of the patient acquired during a second time interval T2, said first time interval T1 and said second time interval T2 differing from each other.

Furthermore, the system comprises means 18 for applying a machine learning algorithm A, in particular an artificial neural network, to the pre-acquired first cardiac current curve data D1 and the pre-acquired at least second cardiac current curve data D2 for classification of the medical relevance of the deviation between the pre-acquired first cardiac current curve data D1 and the pre-acquired at least second cardiac current curve data D2.

The machine learning algorithm A extracts a plurality of features from the pre-acquired first cardiac current curve data D1 and the pre-acquired at least second cardiac current curve data D2.

The machine learning algorithm A then compares this feature set generated based on the pre-acquired first cardiac current curve data D1 with another feature set generated based on the pre-acquired second cardiac current curve data D2 recorded at a previous time. Using machine learning, the differences between respective ECG pairs, i.e., a current vs. a previous ECG of the same patient are compared with each other in order to classify said differences as medically relevant or irrelevant.

The system moreover comprises means 20 for outputting a second data set DS2 comprising at least a first class C1 representing a medically relevant deviation between the first cardiac current curve data D1 and the second cardiac current curve data D2 and/or a second class C2 representing a medically not relevant deviation or no deviation between the first cardiac current curve data D1 and the second cardiac current curve data D2.

The first class C1 representing the medically relevant deviation between the first cardiac current curve data D1 and the second cardiac current curve data D2 comprises a plurality of subclasses C1a, C1b each representing a medical indication, in particular a cardiac disorder.

The second class C2 representing a medically not relevant deviation between the first cardiac current curve data D1 and the second cardiac current curve data D2 comprises changes in position, respiration and/or physical exertion. The second class C2 further represents a case in which there is no deviation between the first cardiac current curve data D1 and the second cardiac current curve data D2 occurs.

The second data set DS2 further comprises a third class C3 representing erroneous cardiac current curve data not suitable for application of the machine learning algorithm A for classification of the medical relevance of the deviation between cardiac current curves.

If the deviation between the pre-acquired first cardiac current curve data D1 and the pre-acquired at least second cardiac current curve data D2 is classified to be a medically relevant deviation according to the first class C1, a notification 11 is sent to a communication device of a health care provider. The medically relevant deviation between the pre-acquired first cardiac current curve data D1 and the pre-acquired at least second cardiac current curve data D2 comprises changes in PQ waves, ST waves, T waves and/or a QRS morphology for ischemia, infarction and/or conduction disorders.

The first data set DS1 further comprises third cardiac current curve data D3 not originating from the patient from which the first cardiac current curve data D1 and the second cardiac current curve data D2 are collected. The first data set DS1 further comprises additional medical parameters 12 comprising a patient activity, a thoracic impedance and/or electrode readings of the implantable medical device 10.

Alternatively or additionally to any embodiment, D1 may be compared with D3. If the machine learning algorithm A detects no medical relevant deviation (=C2), the system may send out a notification 11 to the communication device of a health care provider, that a similar arrhythmia was found in another patient. Preferably the system or the algorithm A is trained accordingly.

The first cardiac current curve data D1 and the second cardiac current curve data D2 comprise a subcutaneous ECG, in particular a wide-field ECG between electrodes and a housing of the implantable medical device 10, a pseudo-ECG between a shock coil and the implantable medical device 10 and/or intracardiac current waveforms. The cardiac current curve data D1, D2 is acquired by the implantable medical device 10 at predetermined intervals and/or on request, and wherein the cardiac current curve data is transmitted to a central server 14 via a patient communication device 16a or smartphone.

Said notification 11 is preferably sent by e-mail. Alternatively, the notification 11 may be sent by text message (SMS) or by means of an in-app notification. Furthermore, the healthcare provider may access the second data set DS2 comprising at least a first class C1 representing a medically relevant deviation between the first cardiac current curve data D1 and the second cardiac current curve data D2 and/or the second class C2 representing a medically not relevant deviation or no deviation between the first cardiac current curve data D1 and the second cardiac current curve data D2 via a front-end application 15 on a suitable communication device 16b such as a smart phone and/or a personal computer.

A beginning of the first time interval T1 differs from a beginning of the second time interval T2 and/or the first time interval T1 ends before a beginning of the second time interval T2.

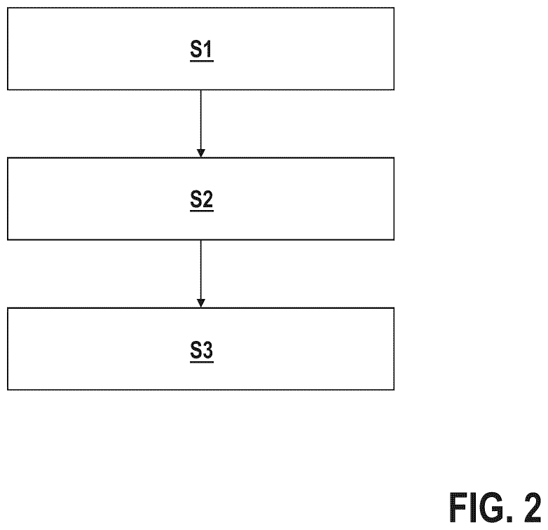

FIG. 2 shows a flowchart of a computer implemented method for providing a trained machine learning algorithm configured to classify a medical relevance of a deviation between cardiac current curves according to the preferred embodiment of the present invention.

The method comprises receiving S1′ a first training data set comprising first cardiac current curve data D1 of a patient acquired during a first time interval T1 and at least second cardiac current curve data D2 of the patient acquired during a second time interval T2 by an implantable medical device 10, said first time interval T1 and said second time interval T2 differing from each other.

Furthermore, the method comprises receiving S2′ a second training data set comprising at least a first class C1 representing a medically relevant deviation between the first cardiac current curve data D1 and the second cardiac current curve data D2 and/or a second class C2 representing a medically not relevant deviation or no deviation between the first cardiac current curve data D1 and the second cardiac current curve data D2.

In addition, the method comprises training S3′ the machine learning algorithm A by an optimization algorithm which calculates an extreme value of a loss function for classification of the first class C1 representing a medically relevant deviation between the first cardiac current curve data D1 and the second cardiac current curve data D2 and/or the second class C2 representing a medically not relevant deviation or no deviation between the first cardiac current curve data D1 and the second cardiac current curve data D2 from the first cardiac current curve data D1 and the second cardiac current curve data D2.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### REFERENCE SIGNS

1 system
10 implantable medical device
11 notification
12 additional medical parameters
14 central server
15 front-end application
16a, 16b communication device
18 means
20 means
A machine learning algorithm
C1 first class
C1a, C1b subclasses
C2 second class
C3 third class
D1 first cardiac current curve data
D2 second cardiac current curve data
D3 third cardiac current curve data
DS1 first data set
DS2 second data set
S1-S3 method steps
S1′-S3′ method steps
T1 first time interval
T2 second time interval

1. Computer-implemented method for classification of a medical relevance of a deviation between cardiac current curves, comprising the steps of:
providing a first data set comprising first cardiac current curve data of a patient acquired during a first time interval and at least second cardiac current curve data of the patient acquired during a second time interval by an implantable medical device, said first time interval and said second time interval differing from each other;
applying a machine learning algorithm to the pre-acquired first cardiac current curve data and the pre-acquired at least second cardiac current curve data for classification of the medical relevance of the deviation between the pre-acquired first cardiac current curve data and the pre-acquired at least second cardiac current curve data; and
outputting a second data set comprising at least a first class representing a medically relevant deviation between the first cardiac current curve data and the second cardiac current curve data and/or a second class representing a medically not relevant deviation or no deviation between the first cardiac current curve data and the second cardiac current curve data.

2. Computer-implemented method of claim 1, wherein the first class representing the medically relevant deviation between the first cardiac current curve data and the second cardiac current curve data comprises a plurality of subclasses each representing a medical indication, in particular a cardiac disorder.

3. Computer-implemented method of claim 1, wherein the second class representing a medically not relevant deviation or no deviation between the first cardiac current curve data and the second cardiac current curve data comprises changes in position, respiration and/or physical exertion.

4. Computer-implemented method of claim 1, wherein the second data set further comprises a third class representing erroneous cardiac current curve data not suitable for application of the machine learning algorithm for classification of the medical relevance of the deviation between cardiac current curves.

5. Computer-implemented method of claim 1, wherein if the deviation between the pre-acquired first cardiac current curve data and the pre-acquired at least second cardiac current curve data is classified to be a medically relevant deviation according to the first class, a notification is sent to a communication device of a health care provider.

6. Computer-implemented method of claim 1, wherein the medically relevant deviation between the pre-acquired first cardiac current curve data and the pre-acquired at least second cardiac current curve data comprises changes in P waves, PQ segment, QRS complex, J point, ST segment, T waves, U waves, TP respectively UP segment and/or a QRS morphology for ischemia, infarction and/or conduction disorders.

7. Computer-implemented method of any claim 1, wherein the first data set further comprises third cardiac current curve data not originating from the patient from which the first cardiac current curve data and the second cardiac current curve data are collected.

8. Computer-implemented method of claim 1, wherein the first data set further comprises additional medical parameters comprising a patient activity, a thoracic impedance and/or electrode readings of the implantable medical device.

9. Computer-implemented method of claim 1, wherein the first cardiac current curve data and the second cardiac current curve data comprise a subcutaneous ECG, in particular a wide-field ECG between electrodes and a housing of the implantable medical device, a pseudo-ECG between a shock coil and the implantable medical device and/or intracardiac current waveforms.

10. Computer-implemented method of claim 1, wherein the cardiac current curve data is acquired by the implantable medical device at predetermined intervals and/or on request, and wherein the cardiac current curve data is transmitted to a central server via a patient communication device or smartphone.

11. Computer-implemented method of claim 1, wherein a beginning of the first time interval differs from a beginning of the second time interval and/or the first time interval ends before a beginning of the second time interval.

12. Computer-implemented method for providing a trained machine learning algorithm-configured to classify a medical relevance of a deviation between cardiac current curves, comprising the steps of:
receiving a first training data set comprising first cardiac current curve data of a patient acquired during a first time interval and at least second cardiac current curve data of the patient acquired during a second time interval by an implantable medical device, said first time interval and said second time interval differing from each other;
receiving a second training data set comprising at least a first class representing a medically relevant deviation between the first cardiac current curve data and the second cardiac current curve data and/or a second class representing a medically not relevant deviation or no deviation between the first cardiac current curve data and the second cardiac current curve data; and
training the machine learning algorithm by an optimization algorithm which calculates an extreme value of a loss function for classification of the first class representing a medically relevant deviation between the first cardiac current curve data and the second cardiac current curve data and/or the second class (representing a medically not relevant deviation between the first cardiac current curve data and the second cardiac current curve data from the first cardiac current curve data and the second cardiac current curve data.

13. System for classification of a medical relevance of a deviation between cardiac current curves, comprising:
an implantable medical device for providing a first data set comprising first cardiac current curve data of a patient acquired during a first time interval and at least second cardiac current curve data of the patient acquired during a second time interval, said first time interval and said second time interval differing from each other;
means for applying a machine learning algorithm to the pre-acquired first cardiac current curve data and the pre-acquired at least second cardiac current curve data for classification of the medical relevance of the deviation between the pre-acquired first cardiac current curve data and the pre-acquired at least second cardiac current curve data; and
means for outputting a second data set comprising at least a first class representing a medically relevant deviation between the first cardiac current curve data and the second cardiac current curve data and/or a second class representing a medically not relevant deviation or no deviation between the first cardiac current curve data and the second cardiac current curve data.

14. Computer program with program code to perform the method of claim 1 when the computer program is executed on a computer.

15. Computer readable data carrier storing a computer program according to claim 14.
